SEQUENCE LISTING (6 sequences)

SEQ ID NO: 1 - DNA, 21 bp, Artificial Sequence
  Myog primer1
tggagctgtatgagacatccc

SEQ ID NO: 2 - DNA, 21 bp, Artificial Sequence
  Myog primer2
tggacaatgctcaggggtccc

SEQ ID NO: 3 - DNA, 20 bp, Artificial Sequence
  Myhc primer1
caagtcatcggtgtttgtgg

SEQ ID NO: 4 - DNA, 20 bp, Artificial Sequence
  Myhc primer2
tgtcgtacttgggcgggttc

SEQ ID NO: 5 - DNA, 20 bp, Artificial Sequence
  Actin primer1
gcctcactgtccaccttcca

SEQ ID NO: 6 - DNA, 22 bp, Artificial Sequence
  Actin primer2
agccatgccaatgttgtctctt

(19) United States
(12) Patent Application Publication
(10) Pub. No.: US 2024/0034836 A1
(43) Pub. Date: Feb. 1, 2024

(54) COATING AGENT, CULTURE SUBSTRATE, AND CELL CULTURE METHOD

(71) Applicants: DENKA COMPANY LIMITED, Tokyo (JP); NATIONAL UNIVERSITY CORPORATION TOKYO MEDICAL AND DENTAL UNIVERSITY, Tokyo (JP)

(72) Inventors: Ruriko AOYAMA SEKIYA, Tokyo (JP); Nobuhiko YUI, Tokyo (JP); Yoshinori ARISAKA, Tokyo (JP); Tetsuya YODA, Tokyo (JP); Hiroki MASUDA, Tokyo (JP)

(73) Assignees: DENKA COMPANY LIMITED, Tokyo (JP); NATIONAL UNIVERSITY CORPORATION TOKYO MEDICAL AND DENTAL UNIVERSITY, Tokyo (JP)

(21) Appl. No.: 18/036,492

(22) PCT Filed: Nov. 10, 2021
(86) PCT No.: PCT/JP2021/041365

§ 371 Date: May 11, 2023

(30) Foreign Application Priority Data
Nov. 13, 2020 (JP) 2020-189697

(51) Int. Cl. C08G 65/334; C12N 5/077

(52) U.S. Cl. CPC C08G 65/334; C12N 5/0658; C12N 2533/30

(57) ABSTRACT

A coating agent including a polyrotaxane represented by Formula (1):

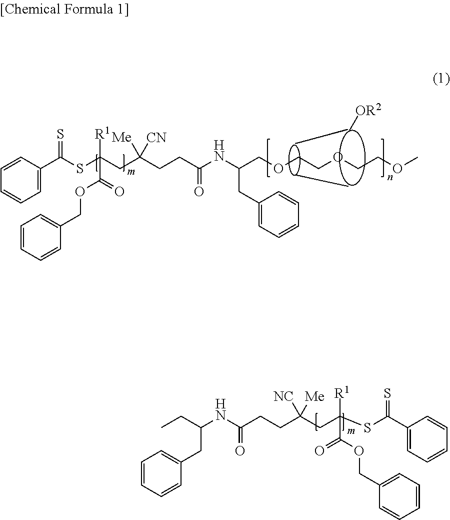

wherein R¹ is a hydrogen atom or a methyl group, m is 1 to 2000, and n is 10 to 500,

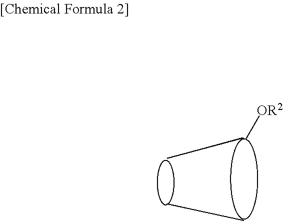

is a cyclodextrin in which at least one hydroxyl group is modified with R², and R² is a C₁₋₆ alkyl group.

### TECHNICAL FIELD

The present invention relates to a coating agent, a culture substrate, and a cell culture method.

### BACKGROUND ART

Cell culture technology is widely used in the pharmaceutical and cosmetic fields and regenerative medicine research, and is an important technology. As environments for cell culture, appropriate culture environments, culture solutions, and culture substrates are required.

Among them, the culture substrate has a role as a scaffold to which cells adhere, and is required to have properties such as being a surface suitable for cell culture or being able to be processed, having no cytotoxicity, being able to be sterilized or maintaining a sterile state, being not deteriorated under culture conditions, and not interfering with observation with a microscope.

Conventionally, as culture substrates, glass products have been widely used, but polystyrene products are now widely used. However, a culture substrate obtained by molding a polystyrene resin is generally subjected to a surface treatment because the surface thereof is hydrophobic and affinity with cells is low. Examples of the surface treatment include a plasma treatment, a corona discharge treatment, an oxidizing agent treatment, and coating with a hydrophilic substance. As the coating agent, there are many coating agents promoting cell adhesion, and for example, Type I collagen, Type IV collagen, gelatin, fibronectin, vitronectin, laminin, matrigel, hydroxyapatite, and the like are known. It is also known that coating with water-soluble elastin promotes differentiation induction of vascular smooth muscle cells or elastin reactive cells.

In recent years, attention has been paid to the fact that the properties (for example, bulk properties such as hardness, and surface properties of coating) of a culture substrate affect the function of cultured cells, and a study for proliferating cells having a desired function has been reported (Non Patent Literatures 1 to 3).

### CITATION LIST

### Patent Literature

Patent Literature 1: JP 2017-23008 A1

### Non-Patent Literature

Non-Patent Literature 1: F. Guilak, D. M. Chohen, B. T. Estes, J. M. Gimble, W. Liedtke, C. S. Chen, Cell. Stem. Cell. 2009, 5, 17.
Non-Patent Literature 2: A. J. Engler, S. Sen, H. L. 1 Sweeney, D. E. Discher, Cell 2006, 126, 677.
Non-Patent Literature 3: A. J. Engler, M. A. Griffin, S. Sen, C. G. Bonnemann, H. L. Sweeney, D. E. Discher, J. Cell. Biol. 2004, 166, 877.

### SUMMARY OF INVENTION

### Technical Problem

However, it is still difficult to finely adjust the bulk properties (hardness or the like) of cell culture substrate in order to obtain cells having a desired function. For example, in the case of myoblasts, it is necessary to control the hardness of the culture substrate to around 11 kPa (9 to 16 kPa). Therefore, an object of the present invention is to provide a cell culture substrate capable of promoting differentiation of cells associated with muscle differentiation, bone differentiation, or adipose differentiation. An object of the present invention is also to provide a cell culture method for promoting differentiation of cells associated with muscle differentiation, bone differentiation, or adipose differentiation.

### Solution to Problem

The present invention provides the following [1] to [16].

[1] A coating agent comprising a polyrotaxane represented by Formula (1):

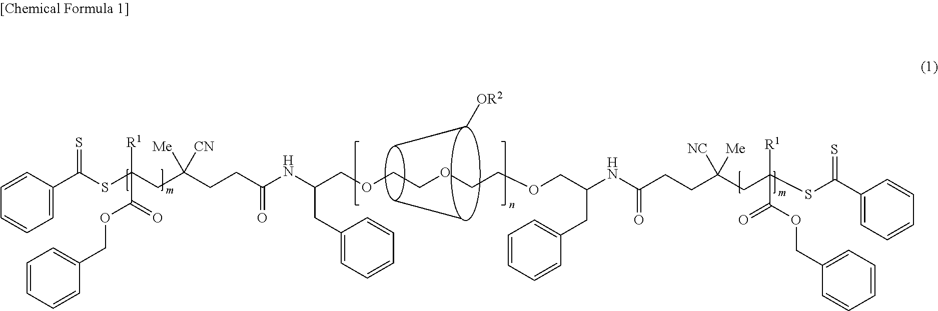

wherein R¹ is a hydrogen atom or a methyl group, m is 1 to 2000, and n is 10 to 500,

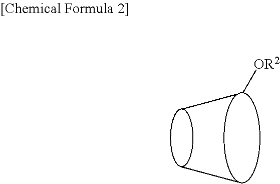

is a cyclodextrin in which at least one hydroxyl group is modified with R², and R² is a C₁₋₆ alkyl group.

[2] The coating agent according to [1], in which the polyrotaxane comprises a cyclodextrin modified with 1 to 18 R²s.

[3] The coating agent according to [1] or [2], in which the polyrotaxane has a number of threaded cyclodextrins of 3 to 220.

[4] A cell culture method for promoting differentiation of cells associated with muscle differentiation, bone differentiation, or adipose differentiation, the method including:
culturing the cells on a surface of a culture substrate,
the culture substrate including a substrate coated with a composition comprising a polyrotaxane represented by Formula (1):

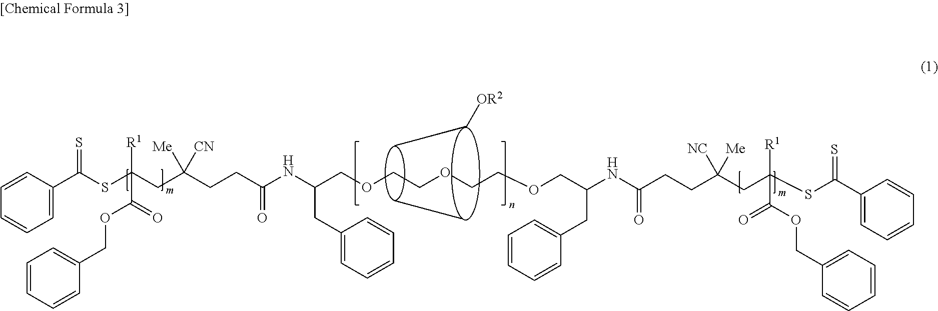

wherein R¹ is a hydrogen atom or a methyl group, m is 1 to 2000, and n is 10 to 500,

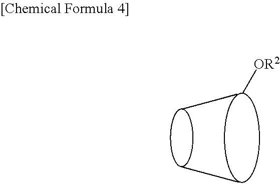

is a cyclodextrin in which at east one hydroxyl group is modified with R², and R² is a C₁₋₆ alkyl group.

[5] The method according to [4], including coating the composition comprising a polyrotaxane represented by Formula (1) onto the surface of the substrate.

[6] The method according to [4] or [5], in which the polyrotaxane comprises a cyclodextrin modified with 1 to 18 R²s.

[7] The method according to any one of [4] to [6], in which the polyrotaxane has a number of threaded cyclodextrins of 3 to 220.

[8] The method according to any one of [4] to [7], in which the cells are preosteoblasts (also referred to as osteoblast-like cells or osteoprogenitor cells), myoblasts, or adipose precursor cells (also referred to as preadipocytes).

[9] The method according to any one of [4] to [7], in which differentiation of the cells includes differentiation of myoblasts into myotubes.

[10] A culture substrate including a substrate coated with a composition comprising a polyrotaxane represented by Formula (1):

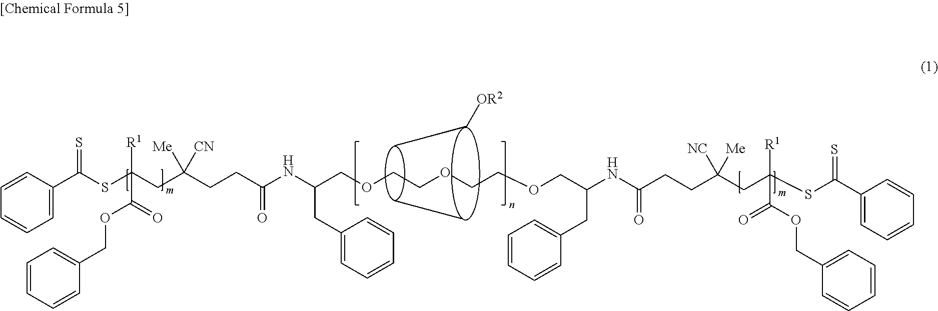

wherein R¹ is a hydrogen atom or a methyl group, m is 1 to 2000, and n is 10 to 500,

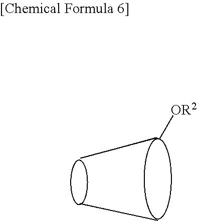

is a cyclodextrin in which at least one hydroxyl group is modified with R², and R² is a C₁₋₆ alkyl group.

[11] The culture substrate according to [10], in which the polyrotaxane comprises a cyclodextrin modified with 1 to 18 R²s.

[12] The culture substrate according to [10] or [11], in which the polyrotaxane has a number of threaded cyclodextrins of 3 to 220.

[13] The culture substrate according to any one of [10] to [12], for promoting differentiation of the cells.

[14] The culture substrate according to [13], in which the cells are preosteoblasts (also referred to as osteoprogenitor cells or osteoblast-like cells), myoblasts, or adipose precursor cells (also referred to as preadipocytes).

[15] The culture substrate according to [13], in which differentiation of the cells includes differentiation of myoblasts into myotubes.

[16] A cell culture method for promoting proliferation of fibroblasts or cells associated with bone differentiation or adipose differentiation, the method including:
culturing the cells on a surface of a culture substrate,
the culture substrate including a substrate coated with a composition comprising a polyrotaxane represented by Formula (1):

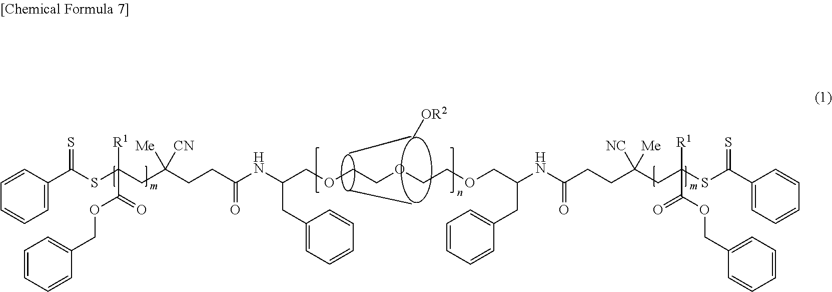

wherein R¹ is a hydrogen atom or a methyl group, m is 1 to 2000, and n is 10 to 500,

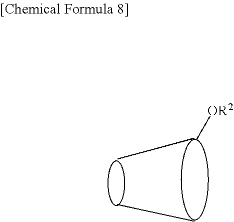

is a cyclodextrin in which at least one hydroxyl group is modified with R², and R² is a C₁₋₆ alkyl group.

### Advantageous Effects of Invention

According to the present invention, the molecular mobility of polyrotaxane can be adjusted by a simple method of adjusting the number of hydroxyl groups modified with R² comprised in a cyclodextrin in the polyrotaxane. In particular, since the cyclodextrin is modified in the final step, it is also easy to readjust the molecular mobility. By using a substrate applied with the coating comprising a polyrotaxane (that is, a culture substrate), differentiation of cells associated with muscle differentiation, bone differentiation, or adipose differentiation can be promoted. The differentiation promoted by the present invention is differentiation at a stage after the lineage of differentiation is determined, for example, the differentiation is differentiation of myoblasts into myotubes via myocytes in the case of muscle differentiation, differentiation of preosteoblasts into osteocytes via osteoblasts (for example, immature osteoblasts and mature osteoblasts) or differentiation of immature chondrocytes into mature chondrocytes in the case of bone differentiation, and differentiation of adipose precursor cells into adipocytes in the case of adipose differentiation. According to the present invention, all or part of the differentiation can be promoted.

According to the coating agent of the present invention, not only differentiation of cells associated with muscle differentiation, bone differentiation, or adipose differentiation is promoted, but also the cells (for example, fibroblasts, preosteoblasts, or adipose precursor cells) can easily adhere to the culture substrate, and the proliferation of the cells themselves can be promoted.

These effects can be exhibited by applying a coating comprising a polyrotaxane having molecular mobility adjusted to exert a desired function without being limited by the bulk properties (particularly, physical properties such as hardness) of the substrate itself. Therefore, it may be freely combined with existing medical materials, medical equipment, medical devices, and the like. Conventionally, it has been known that promotion of differentiation is observed depending on the bulk properties (for example, hardness) of the substrate itself, but by applying the coating of the present invention, it is possible to promote the differentiation of a wide range of desired cells regardless of the bulk properties of the substrate itself.

### BRIEF DESCRIPTION OF DRAWINGS

FIG. 1 shows a ¹H-NMR spectrum of a polyrotaxane of Comparative Example 1.

FIG. 2 shows a ¹H-NMR spectrum of a polyrotaxane of Example 3.

FIG. 3(A) is a graph showing a contact angle with respect to a water droplet, FIG. 3(B) is a graph showing a contact angle of water calculated from a value of a contact angle with respect to a bubble, and FIG. 3(C) is a graph showing a contact angle hysteresis value.

FIG. 4 is a graph showing comparison of the expression levels of differentiation marker genes (Myog and Myhc) when polyrotaxanes of Reference Example 2, Comparative Example 1, and Examples 1 to 3 are used.

FIGS. 5(A), 5(B), and 5(C) are graphs showing changes in cell density of BALB/3T3 cells, MC3T3-E1 cells, and MC3T3-G2/PA6 cells, respectively.

FIG. 6(A) is an image of cells stained with Alizarin Red S, and FIG. 6(B) is a graph obtained by quantifying a stained region.

FIG. 7(A) is an image of cells stained with Oil Red O, and FIG. 7(B) is a graph obtained by quantifying a stained region.

### DESCRIPTION OF EMBODIMENTS

### First Embodiment

A first embodiment of the present invention is a coating agent comprising a polyrotaxane represented by Formula (1).

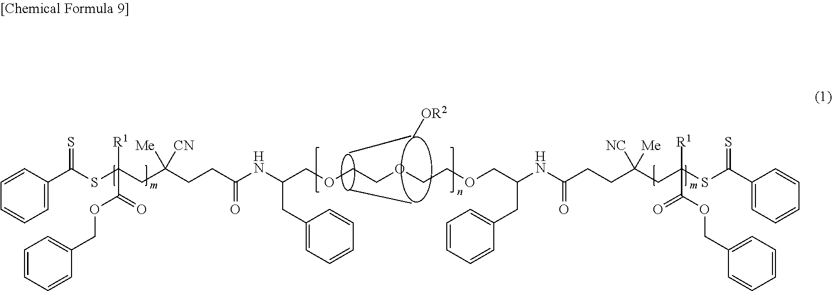

wherein R¹ is a hydrogen atom or a methyl group, m is 1 to 2000, and n is 10 to 500,

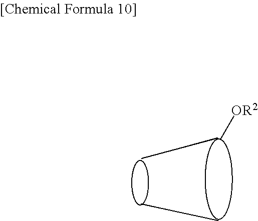

is a cyclodextrin in which at least one hydroxyl group is modified with R², and R² is a C₁₋₆ alkyl group. At least one of the cyclodextrins is comprised in one molecule of the polyrotaxane.

The polyrotaxane represented by Formula (1) includes an axial molecule (linear polymer) and a modified α-cyclodextrin (αCD, cyclic molecule), and the axial molecule threads at least one modified cyclodextrin and is capped at both ends thereof.

The axial molecule is represented by Formula (2) below, has a polyethylene glycol structure at the center of the molecule, and has a poly(meth)acrylate structure capped with a phenyldithioester group at the terminal. In the formula, R¹ is a hydrogen atom or a methyl group. The cyclodextrin is threaded to the polyethylene glycol structure portion.

Each R¹ may be the same as or different from each other. R¹ is preferably a methyl group.

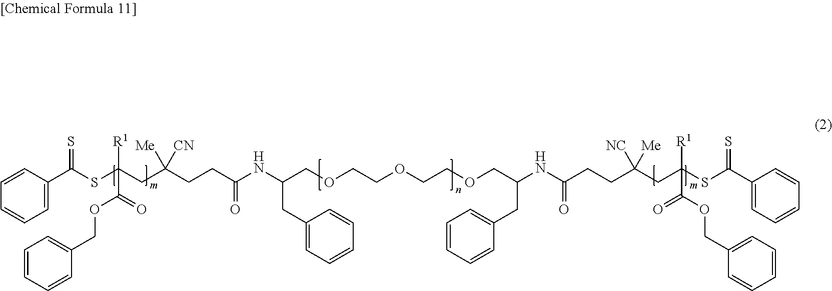

The polyethylene glycol structure may include ethylene glycol as a monomer unit and have a molecular length capable of threading at least one cyclodextrin. The number of ethylene glycol units forming the polyethylene glycol structure may be 20 to 1000, and is preferably 85 to 800 and more preferably 100 to 700. In the formula, n may be 10 to 500, and is preferably 43 to 400 and more preferably 50 to 350.

The poly(meth)acrylate structure includes benzyl (meth)acrylate as a monomer unit. In the present specification, the term “(meth)acrylate” means both “acrylate” and “methacrylate”. The present inventors consider that since the poly(meth)acrylate structure has an action of enhancing adhesion to a substrate such as polystyrene, the polyethylene glycol moiety threading the cyclodextrin is separated from the substrate in a loop shape. The number m of benzyl (meth)acrylate units forming the poly(meth)acrylate structure may be 1 to 2000, and is preferably 20 to 1000 and more preferably 30 to 500, per polybenzyl (meth)acrylate at one terminal of the triblock copolymer. m may be the same as or different from each other.

In the modified cyclodextrin used in the present embodiment, at least one hydroxyl group of glucose constituting the cyclodextrin is modified with R². The cyclodextrin may be any of α-cyclodextrin, β-cyclodextrin, γ-cyclodextrin, and combinations thereof. A preferred cyclodextrin is α-cyclodextrin. The following is a figure schematically showing a cyclodextrin modified with R². “OR²” in the figure indicates that a hydroxyl group in glucose constituting the cyclodextrin is modified with R². In the figure, only one “OR²” is shown, but it should not be interpreted restrictively that only one hydroxyl group is modified with R².

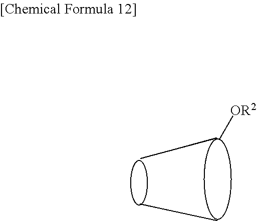

In the polyrotaxane, at least one modified cyclodextrin is threaded by an axial molecule. The number of cyclodextrins per polyrotaxane molecule can be independently determined and need not be uniquely determined by the molecular length of the polyethylene glycol structure. Stoichiometrically, since the α-cyclodextrin can include two units of ethylene glycol, which is a repeating unit of polyethylene glycol, the number of cyclic molecules has an upper limit depending on the molecular weight of the linear polymer to be used. For example, the number of cyclodextrins per polyethylene glycol molecule having a number average molecular weight of 20000 may be 3 to 220, and is preferably 5 to 150, more preferably 5 to 120, and particularly preferably 5 to 100. However, the length (total length when the polyrotaxane has a plurality of cyclodextrins) of the axial molecule of the cyclodextrin in the main chain direction does not exceed the molecular length of polyethylene glycol.

The number of hydroxyl groups modified with R² in the cyclodextrin may be 1 to 18, and is preferably 5 to 17 and more preferably 7 to 16, with respect to one cyclodextrin. In particular, in the case of differentiation of myoblasts, the number of hydroxyl groups is preferably 2 to 17, more preferably 3 to 16, and further preferably 5 to 15. When the number of hydroxyl groups modified with R² is in the above range, the effect of promoting proliferation and differentiation of cells associated with muscle differentiation, bone differentiation, or adipose differentiation is more excellent. When the number of hydroxyl groups modified with R² is 17 or less, solubility in an organic solvent such as dimethyl sulfoxide (DMSO) is more excellent. When the number of hydroxyl groups modified with R² is 2 or more, the amount of change in molecular mobility increases. Since the molecular mobility of the polyrotaxane can be adjusted by the number of hydroxyl groups modified with R², a polyrotaxane surface having molecular mobility suitable for each of muscle differentiation, bone differentiation, and adipose differentiation can be prepared.

R² is a C₁₋₆ alkyl group, and examples thereof include a methyl group, an ethyl group, a 1-propyl group, a 2-propyl group, a 1-butyl group, a 2-butyl group, a tert-butyl group, a 1-pentyl group, a 2-pentyl group, a 3-pentyl group, a neopentyl group, a 1-hexyl group, a 2-hexyl group, and a 3-hexyl group. R² is preferably a methyl group or an ethyl group, and R² is more preferably a methyl group. Each R² may be the same as or different from each other.

The polyrotaxane includes a structure in which an axial molecule threads at least one cyclodextrin. Each cyclodextrin can move along the main chain direction of the axial molecule and rotate about the main chain of the axial molecule. Such structural properties are referred to as molecular mobility. The molecular mobility can vary, for example, according to the number of cyclodextrins, and the number of substituents added to glucose constituting the cyclodextrin constituting therewith.

The molecular mobility can be evaluated by coating the surface of a culture substrate, then measuring the static contact angle of the coating surface using a contact angle meter, and using a droplet method and a captive bubble method. For the evaluation of molecular mobility, for example, methods described in Soft Matter, 2012, 8, 5477 (Ji-Hun Seo et al.), Adv. Healthcare Mater. 2015, 4, 215-222, and the like may be referred to. Specifically, a contact angle hysteresis value of the coating surface can be calculated from the difference in contact angle between the droplet and the bubble. As a control, comparison with the effect on a culture substrate coated with DMSO may be conducted.

The molecular mobility can be adjusted by changing the number of threaded cyclodextrins and/or the number of hydroxyl groups modified with R² in the cyclodextrin. For example, when the number of threaded cyclodextrins is 20 to 120, it can be more suitable for promoting muscle differentiation. When the number of threaded cyclodextrins is 50 to 200, bone differentiation can be more suitably promoted, and when the number of threaded cyclodextrins is 3 to 40, adipose differentiation can be more suitably promoted. When the number of threaded cyclodextrins is large, the cyclodextrin is easily purified by reprecipitation.

The content of the polyrotaxane may be 0.0005 to 5 mass %, preferably 0.01 to 1 mass %, and more preferably 0.02 to 0.5 mass %, based on the mass of the coating agent.

The coating agent of the present embodiment may comprise a solvent and an optional additive in addition to the above-described polyrotaxane. Examples of the solvent include dimethyl sulfoxide (DMSO), tetrahydrofuran (THF), N,N-dimethylformamide (DMF), methanol, 2-propanol, chloroform, and methylene chloride. Examples of such an additive include an antioxidant.

The coating agent of the present embodiment is used for coating a surface of a substrate that can be used as a culture substrate. The substrate that can be used as a culture substrate may be a substrate well known to those skilled in the art. Examples of the material for the substrate include glass, polystyrene, polypropylene, polyethylene, polyolefin, polycarbonate, and an acrylic block copolymer (BCF).

According to the coating agent of the present embodiment, by adjusting the number of hydroxyl groups modified with R² in glucose constituting the cyclodextrin, it is possible to adjust differentiation control (promotion or suppression) of cells associated with muscle differentiation, bone differentiation, or adipose differentiation and proliferation control (promotion or suppression) of cells associated with muscle differentiation, bone differentiation, or adipose differentiation without depending on the bulk properties of the substrate itself. More specifically, the expression level of a differentiation marker gene (for example, Myog and Myhc), which is an index for differentiation of myoblasts into myotubes, increases. The number of bone nodules (stained with alizarin red dye) formed in the differentiation of preosteoblasts into osteoblasts increases. In differentiation of adipose precursor cells into adipocytes, lipid droplets (stained with oil red dye) that accumulate in cells increase.

The polyrotaxane can be produced with reference to examples, and may be produced as follows. Hydroxyl groups at both ends of polyethylene glycol having a desired length are converted into leaving groups (for example, halogenation, methanesulfonylation, or toluenesulfonylation), and etherified with phenylalaninol to obtain a diamine. The obtained diamine is mixed with a cyclodextrin to obtain a pseudo-rotaxane. At this time, the number of threaded cyclodextrins can be adjusted by adjusting the amount of cyclodextrin per diamine molecule. Subsequently, the mixture is reacted with CPADB and DMT-MM to cap the cyclodextrin so as not to be removed from the axial molecule, and then benzyl methacrylate (corresponding to a (meth)acrylate structure) is introduced at both terminals as an anchoring segment by a reversible addition-fragmentation chain transfer polymerization reaction (RAFT polymerization reaction). Finally, a polyrotaxane can be obtained by reacting an alkylating agent (for example, alkyl iodide) in the presence of sodium hydroxide to modify a hydroxyl group to a desired degree with R².

### Second Embodiment

A second embodiment of the present invention is a cell culture method for promoting differentiation of cells associated with muscle differentiation, bone differentiation, or adipose differentiation, the method including: culturing the cells on a surface of a culture substrate, the culture substrate including a substrate coated with a composition comprising a polyrotaxane represented by Formula (1).

In the present embodiment, as the “polyrotaxane represented by Formula (1)” and the “cells associated with muscle differentiation, bone differentiation, or adipose differentiation”, those described in the first embodiment can be referred to, and as the “composition comprising a polyrotaxane represented by Formula (1)”, the coating agent described in the first embodiment can be used.

In the cell culture method of the present embodiment, as the culture substrate, a substrate coated with a composition comprising a polyrotaxane represented by Formula (1) is used. Cells are cultured by adhering the cells to the surface of the coated substrate.

The culture substrate is obtained by applying a coating comprising a polyrotaxane represented by Formula (1) to a substrate. The substrate may be a substrate well known to those skilled in the art. Examples of the material for the substrate include glass, polystyrene, polypropylene, polyethylene, polyolefin, polycarbonate, and an acrylic block copolymer (BCF). The substrate may be a commercially available glass substrate or plastic substrate.

In the cell culture method of the present embodiment, the cells to be cultured are seeded on the coating surface provided on the substrate. Thereafter, the medium is added so that the cells are immersed, and the cells are cultured. The medium may be replaced with a new medium as necessary. A step of injecting a medium for proliferation before differentiating the cells to proliferate the seeded cells may be provided. In this case, after the cells are proliferated until a sufficient number of cells is obtained, the medium for proliferation is replaced with a medium for differentiation. As the medium for proliferation and the medium for differentiation, a medium well known to those skilled in the art can be used.

Media used for differentiation of myoblasts, preosteoblasts, and adipose precursor cells are, for example, as follows.
Medium for differentiation of myoblasts:
DMEM, 1% FBS, 100 mg/mL streptomycin, and 1% penicillin
Medium for differentiation of preosteoblasts:
α-MEM, 10% FBS, 100 U/mL penicillin, 100 mg/mL streptomycin, 50 μg/mL magnesium L-ascorbic acid phosphate, and 10 mM disodium β-glycerophosphate pentahydrate
Medium for differentiation of adipose precursor cells:
α-MEM, 10% FBS, 100 U/mL penicillin, 100 mg/mL streptomycin, 0.5 mM IBMX, and 0.25 mM dexamethasone

The cell culture environment can be arbitrarily set under the conditions well known to those skilled in the art.

The effect of promoting differentiation of cells associated with muscle differentiation, bone differentiation, or adipose differentiation can be evaluated by measuring a change in the expression level of a differentiation marker. For example, in the case of myoblasts, Myog (myogenin), Myhc (myosin heavy chain), MyoD, Myf5, and MRF5 are exemplified. In the case of preosteoblasts, Runx2, alkaline phosphatase, osteocalcin, osteopontin, bone sialoprotein, and Type I collagen are exemplified. In the case of adipose precursor cells, PPARγ, C/EBPα, and aP2 are exemplified. In this case, the expression level is preferably corrected by the expression level of a housekeeping gene such as β-actin or GAPDH (glyceraldehyde-3-phosphate dehydrogenase). The effect may be determined by cell staining with Alizarin Red S (staining a calcified bone nodule) in the case of preosteoblasts and by cell staining with Oil Red O (staining an intracellular lipid droplet) in the case of adipose precursor cells. When the differentiation is statistically significantly promoted as compared with the case of culturing using an uncoated culture substrate (a glass product or a polystyrene product), it can be determined that the effect of promoting the differentiation of the cells is attained.

The cell culture method of the present embodiment may include coating the composition comprising a polyrotaxane represented by Formula (1) onto the surface of the substrate.

The composition comprising a polyrotaxane represented by Formula (1) can be coated on a substrate. The coating method is not particularly limited, and examples thereof include casting, spin coating, gravure coating, die coating, knife coating, bar coating, blade coating, and roll coating. A preferred coating method is casting.

### Third Embodiment

A third embodiment of the present invention is a culture substrate including a substrate coated with a composition comprising a polyrotaxane represented by Formula (1).

In the present embodiment, as the “polyrotaxane represented by Formula (1)”, those described in the first embodiment can be referred to, and as the “composition comprising a polyrotaxane represented by Formula (1)”, the coating agent described in the first embodiment can be used. As the coating method, the method described in the second embodiment can be referred to.

The coated substrate (culture substrate) of the present embodiment is particularly suitable as a culture substrate for promoting differentiation of cells associated with muscle differentiation, bone differentiation, or adipose differentiation. Specific examples of differentiation of cells associated with muscle differentiation, bone differentiation, or adipose differentiation include differentiation of myoblasts into myotubes, differentiation of myoblasts into myocytes, differentiation of myocytes into myotubes, differentiation of preosteoblasts into osteoblasts, differentiation of osteoblasts into osteocytes, and differentiation of adipose precursor cells into adipocytes.

According to the culture substrate of the present embodiment, by adjusting the number of hydroxyl groups modified with R² in glucose constituting the cyclodextrin, it is possible to adjust differentiation control (promotion or suppression) of cells associated with muscle differentiation, bone differentiation, or adipose differentiation and proliferation control (promotion or suppression) of cells associated with muscle differentiation, bone differentiation, or adipose differentiation without depending on the bulk properties of the substrate itself. More specifically, the expression level of a differentiation marker gene (for example, Myog and Myhc), which is an index for differentiation of myoblasts into myotubes, increases. The number of bone nodules (stained with alizarin red dye) formed in the differentiation of preosteoblasts into osteoblasts increases. In differentiation of adipose precursor cells into adipocytes, lipid droplets (stained with oil red dye) that accumulate in cells increase.

### Fourth Embodiment

A fourth embodiment of the present invention is a cell culture method for promoting proliferation of fibroblasts or cells associated with bone differentiation or adipose differentiation, the method including: culturing the cells on a surface of a substrate coated with a composition comprising a polyrotaxane represented by Formula (1).

In the present embodiment, as the polyrotaxane represented by Formula (1), the cells associated with bone differentiation, and the cells associated with adipose differentiation, those described in the first embodiment can be referred to, and as the “composition comprising a polyrotaxane represented by Formula (1)”, the coating agent described in the first embodiment can be used.

In the cell culture method of the present embodiment, as the culture substrate, a substrate coated with a composition comprising a polyrotaxane represented by Formula (1) can be used. Cells are cultured by adhering the cells to the surface of the coating.

The culture substrate is obtained by applying a coating comprising a polyrotaxane represented by Formula (1) to a substrate. The substrate may be a substrate well known to those skilled in the art. Examples of the material for the substrate include glass, polystyrene, polypropylene, polyethylene, polyolefin, polycarbonate, and an acrylic block copolymer (BCF). The substrate may be a commercially available glass substrate or plastic substrate.

The cell culture method of the present embodiment may include coating the composition comprising a polyrotaxane represented by Formula (1) onto the surface of the substrate.

The composition comprising a polyrotaxane represented by Formula (1) can be coated on a substrate. The coating method is not particularly limited, and examples thereof include casting, spin coating, gravure coating, die coating, knife coating, bar coating, blade coating, and roll coating. A preferred coating method is casting.

In the cell culture method of the present embodiment, the cells to be cultured are seeded on the coating surface provided on the substrate. Thereafter, the medium is added so that the cells are immersed, and the cells are cultured. The medium may be replaced with a new medium as necessary. As the medium for proliferation, a medium well known to those skilled in the art can be used. After the cells are proliferated until a sufficient number of cells is obtained, the medium for proliferation may be replaced with a medium for differentiation.

Medium for proliferation of fibroblasts:
DMEM, 10% FBS, 100 U/mL penicillin, and 100 mg/mL streptomycin

Medium for proliferation of preosteoblasts:
α-MEM, 10% FBS, 100 U/mL penicillin, and 100 mg/mL streptomycin

Medium for proliferation of adipose precursor cells:
α-MEM, 10% FBS, 100 U/mL penicillin, and 100 mg/mL streptomycin

Medium for differentiation of preosteoblasts:
α-MEM, 10% FBS, 100 U/mL penicillin, 100 mg/mL streptomycin, 50 μg/mL magnesium L-ascorbic acid phosphate, and 10 mM disodium β-glycerophosphate pentahydrate

Medium for differentiation of adipose precursor cells:
α-MEM, 10% FBS, 100 U/mL penicillin, 100 mg/mL streptomycin, 0.5 mM IBMX, and 0.25 mM dexamethasone

According to the cell culture method of the present embodiment, proliferation of fibroblasts or cells associated with bone differentiation or adipose differentiation can be accelerated.

### EXAMPLES

Hereinafter, the present invention will be described in more detail with reference to Examples. However, the present invention is not limited thereto. The abbreviations used in the examples are conventional abbreviations well known to those skilled in the art, and the meanings of some abbreviations are shown below.
αCD: α-cyclodextrin
CPADB: 4-cyanopentanoic acid dithiobenzoate
DMEM: Dulbecco's modified Eagle medium
DMF: N,N-dimethylformamide
DMSO: dimethyl sulfoxide
DMT-MM: 4-(4,6-dimethoxy-1,3,5-triazine-2-yl)-4-methylmorpholinium chloride
EDTA: ethylenediaminetetraacetic acid
FBS: fetal bovine serum
IBMX: 3-isobutyl-1-methylxanthine
α-MEM: α-minimum essential medium
MeOH: methanol
MsCl: methanesulfonic acid chloride
PBS: phosphate buffered saline
PEG: polyethylene glycol
TCPS: polystyrene for cell culture
TEA: triethylamine
THF: tetrahydrofuran
¹H-NMR: proton nuclear magnetic resonance spectrometry

1. Synthesis of Polyrotaxane

### Example 1

A method for synthesizing a polyrotaxane is shown below.

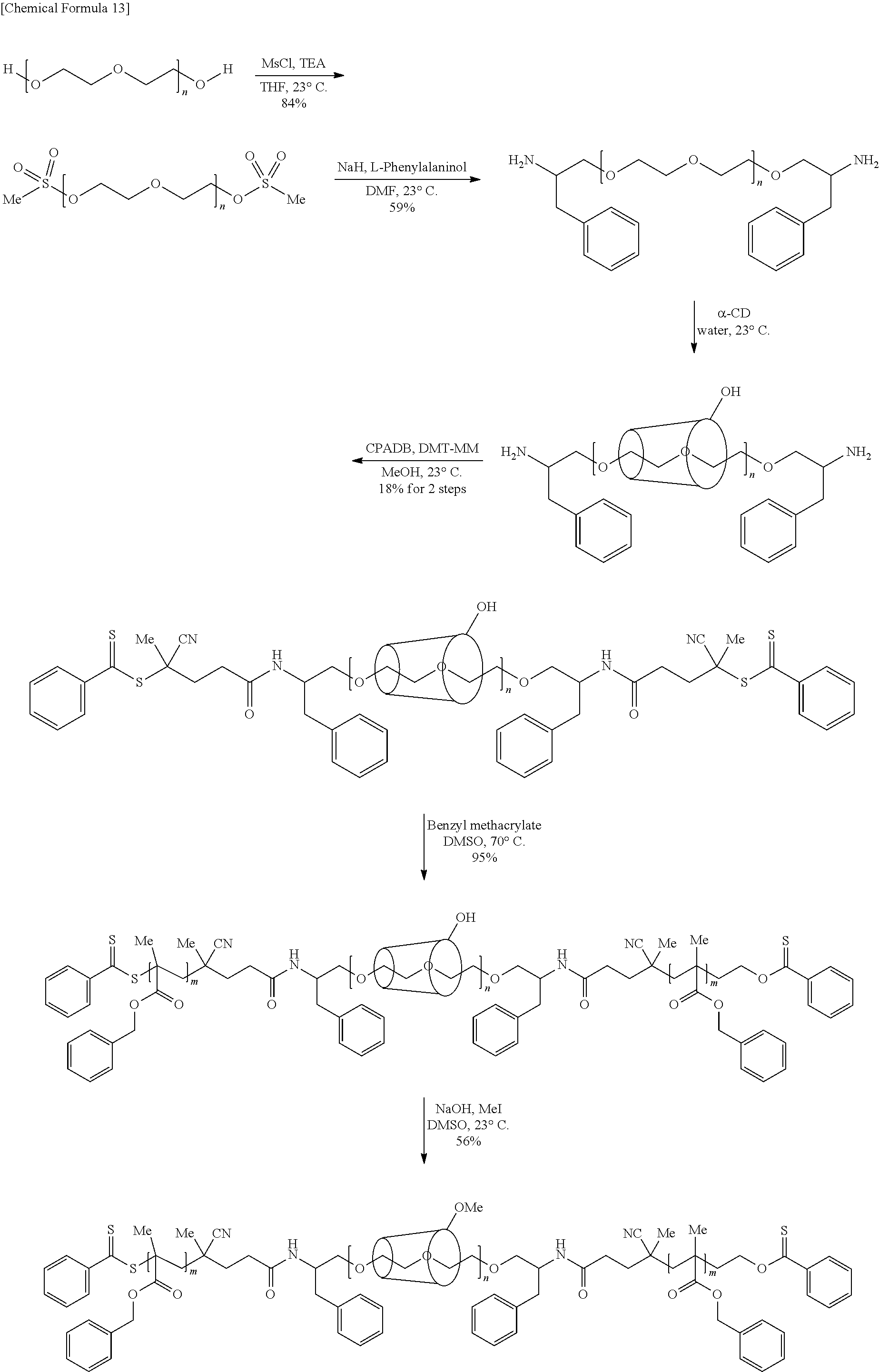

### Step 1: Synthesis of α,ω-Bismesyl Polyethylene Glycol

Polyethylene glycol having a number average molecular weight of 20000 (25.0 g, 1.25 mmol) and TEA (5.3 mL, 37.5 mmol) were dissolved in anhydrous THE (130 mL), MsCl (2.0 mL, 25.0 mmol) was added dropwise, and the mixture was stirred at 23° C. After 5 hours, the reaction solution was filtered, the filtrate was precipitated with diethyl ether, and the precipitate was collected as a solid. The obtained precipitate was dried under reduced pressure to obtain α,ω-bismesyl polyethylene glycol (21.1 g, yield: 840%).

### Step 2: Synthesis of Bis(2-Amino-3-Phenylpropyl)Polyethylene Glycol

L-Phenylalaninol (1.49 g, 9.85 mmol) and sodium hydride (0.971 g, 6000 mineral oil) were dissolved in anhydrous DMF (86 mL) under a nitrogen atmosphere, and was added. To this mixed liquid, α,ω-bismesyl polyethylene glycol (20.0 g, 0.992 mmol) was added, and the mixture was stirred at 23° C. After 24 hours, the reaction solution was filtered, the filtrate was precipitated with diethyl ether, and the precipitate was collected as a solid. The obtained precipitate was dried under reduced pressure to obtain bis(2-amino-3-phenylpropyl)polyethylene glycol (11.8 g, yield: 59%).

### Step 3: Synthesis of Pseudo-Polyrotaxane

Bis(2-amino-3-phenylpropyl)polyethylene glycol (10.1 g, 0.499 mmol) was dissolved in water (50 mL), a saturated aqueous solution (380 mL) of αCD (55.2 g, 56.6 mmol) was added thereto, and the mixture was stirred at 23° C. After 19 hours, the reaction solution was centrifuged to collect a precipitate, and freeze-dried for 9 days to obtain a pseudo-polyrotaxane as a crude product.

### Step 4: Synthesis of Polyrotaxane PRX-CPADB

CPADB (5.50 g, 19.7 mmol) and DMT-MM (5.50 g, 19.9 mmol) were dissolved in methanol (500 mL), and the pseudo-polyrotaxane obtained above was added to the reaction solution at 23° C., followed by stirring. After 1 day, the crude product was washed with methanol, reprecipitated with hydrous DMSO, centrifuged and lyophilized for 9 days to obtain the polyrotaxane PRX-CPADB (11.9 g, 2-step yield: 18%) as a powder. The structure of polyrotaxane PRX-CPADB was confirmed by ¹H-NMR (solvent: DMSO-d₆). The number of threaded αCD was determined by ¹H-NMR (solvent: D₂O).

¹H NMR (500 MHz, DMSO-d₆) δ 3.12-3.91 (m, PEG backbone and H2, H3, H4, H5, and H6 protons of αCD), 4.43 (m, OH6 of αCD), 4.80 (m, H1 of αCD), 5.49 (m, OH3 of αCD), 5.65 (in, OH2 of αCD), 7.17 (t, aromatics of phenylalanyl group), 7.25 (t, aromatics of phenylalanyl group), 7.52 (t, aromatics of CPADB group), 7.70 (t, aromatics of CPADB), and 7.91 (t, aromatics of CPADB).

### Step 5: Synthesis of Polyrotaxane PRX-PBzMA

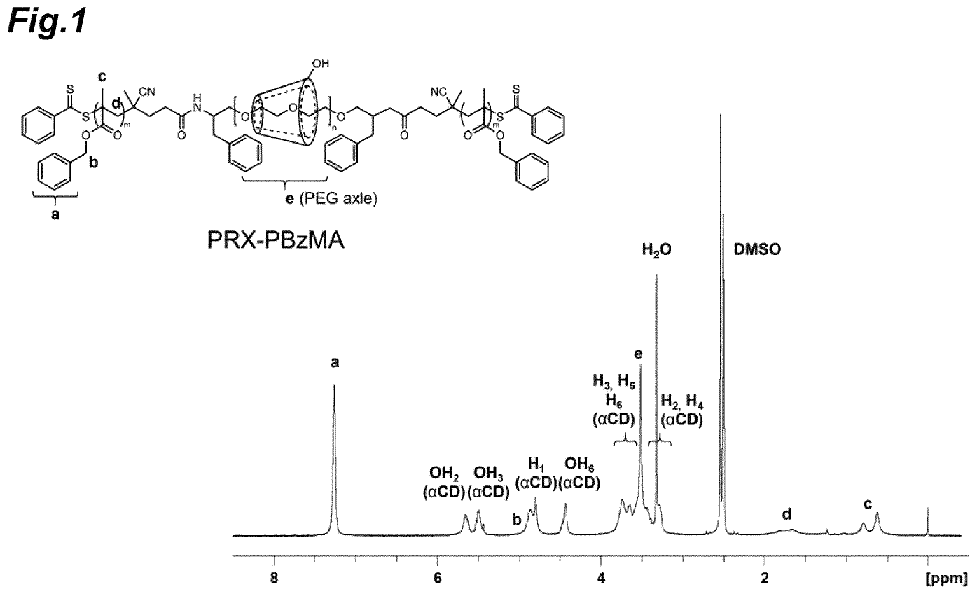

Polyrotaxane PRX-CPADB (1.48 g, 13.7 μmol) was dissolved in anhydrous DMSO (8.3 mL), and benzyl methacrylate (0.749 g, 4.25 mmol) and 4,4′-azobis(4-cyanovaleric acid) (1.55 mg, 5.54 μmol) were added to this mixed liquid. After degassing by FPT cycle (Freeze-Pump-Thaw cycle), the mixture was stirred at 70° C. After 1 day, the crude product was precipitated with diethyl ether and dried under reduced pressure to obtain the polyrotaxane PRX-PBzMA (2.10 g, yield: 95%). ¹H-NMR of the polyrotaxane is shown in FIG. 1.

¹H NMR (500 MHz, DMSO-d₆) δ 0.47-0.91 (m, —CH(—CH3)-CH2- of PBzMA), 1.47-1.98 (m, —CH(—CH3)-CH2- of PBzMA), 3.24-3.81 (m, PEG backbone and H2, H3, H4, H5, and H6 protons of αCD), 4.43 (m, OH6 of αCD), 4.80 (m, H1 of αCD), 4.86 (m, —CH2-Ph of PBzMA), 5.49 (m, OH3 of αCD), 5.65 (m, OH2 of αCD), and 7.26 (m, aromatics of PBzMA).

### Step 6: Synthesis of Polyrotaxane of Example 3

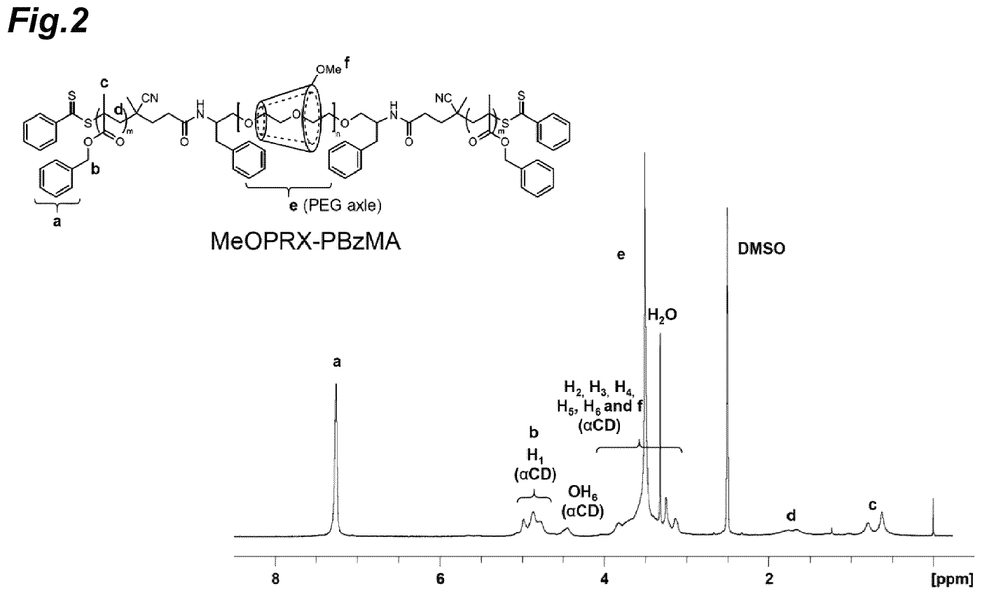

Polyrotaxane PRX-PBzMA (0.2 g, 1.23 μmol) was dissolved in anhydrous DMSO (11 mL), sodium hydroxide (0.236 g, 5.90 mmol) and iodomethane (122 μL, 1.97 mmol) were added thereto, and the mixture was stirred at 23° C. After 1 hour, methanol was added to stop the reaction, and the mixture was dialyzed for 3 days to obtain a polyrotaxane (130 mg, yield: 56%) of Example 3. ¹H-NMR of the polyrotaxane is shown in FIG. 2.

¹H NMR (500 MHz, DMSO-d₆) δ 0.47-0.91 (m, —CH(—CH3)-CH2- of PBzMA), 1.47-1.98 (m, —CH(—CH3)-CH2- of PBzMA), 3.06-4.04 (m, PEG backbone, H2, H3, H4, H5, and H6 protons of αCD, and —OCH3 of αCD), 4.46 (m, OH6 of αCD), 4.57-5.20 (m, H1 of αCD and —CH2-Ph of PBzMA), and 7.26 (m, aromatics of PBzMA).

### Example 1

A polyrotaxane of Example 1 was obtained in the same manner as in Example 3, except that the ratio of the number of moles of iodomethane with respect to the number of moles of all hydroxyl groups in the polyrotaxane PRX-PBzMA was changed to 0.11 in Step 6.

### Example 2

A polyrotaxane of Example 2 was obtained in the same manner as in Example 3, except that the ratio of the number of moles of iodomethane with respect to the number of moles of all hydroxyl groups in the polyrotaxane PRX-PBzMA was changed to 0.44 in Step 6.

### Comparative Example 1

The polyrotaxane PRX-PBzMA obtained in Step 5 was used as Comparative Example 1.

The chemical compositions of the polyrotaxanes of Examples 1 to 3 and Comparative Example 1 are shown in Table 1. Each number average molecular weight was calculated based on an NIR spectrum. The number in parentheses is the number of methylated hydroxyl groups (methoxy groups) per cyclodextrin.

TABLE 1

| | Number | | | Number | |
|---|---|---|---|---|---|
| | average | | Number | average | |
| | molecular | Number | average | molecular | Number of |
| | weight of | of | molecular | weight of | methylated |
| | PEG | threaded | weight of | axial | hydroxyl |
| | moiety | αCD | polyrotaxane | molecule | groups |
| Compar- | 20000 | 89.8 | 108200 | 54400 | 0 (0) |
| ative | | | | | |
| Example | | | | | |
| 1 | | | | | |
| Example | 20000 | 89.8 | 108200 | 54400 | 165 (2) |
| 1 | | | | | |
| Example | 20000 | 89.8 | 108200 | 54400 | 592 (7) |
| 2 | | | | | |
| Example | 20000 | 89.8 | 108200 | 54400 | 1342 (15) |
| 3 | | | | | |

2. Evaluation of Molecular Mobility

The coatings comprising polyrotaxane of Examples 1 to 3 were applied to the surface of polystyrene for cell culture (TCPS). The static contact angle of the coating surface was measured using a contact angle meter (trade name: DM-501, manufactured by Kyowa Interface Science Co., Ltd. by both of a droplet method and a captive bubble method. A contact angle hysteresis value of each surface was calculated from a difference between a contact angle with respect to a droplet and a contact angle of water obtained from a contact angle of a bubble.

The surface of the used TCPS was coated with 30 μL of DMSO, dried, and used as a control (Reference Example 1). All the measurements were performed on three different surfaces, and the average value thereof was calculated. Statistical analysis was performed using one-way analysis of variance (ANOVA) and the Tukey HSD method. Statistical significance was indicated with ***p<0.001, **p<0.01, and *p<0.05.

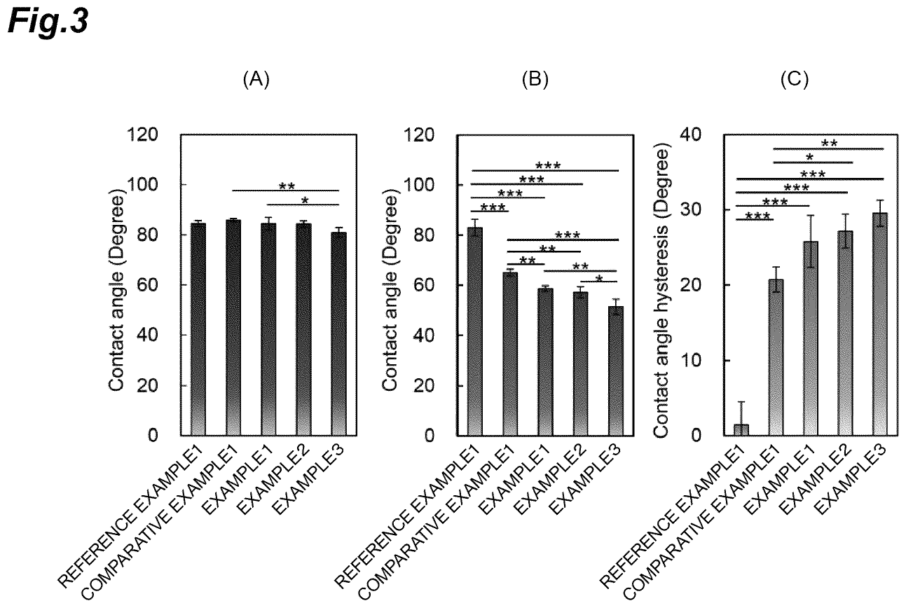

The results are shown in FIG. 3. FIG. 3(A) is a graph showing a contact angle with respect to a water droplet on each surface, FIG. 3(B) is a graph showing a contact angle of water obtained from a contact angle with respect to a bubble on each surface, and FIG. 3(C) is a graph showing a contact angle hysteresis value on each surface. As the number of methylated hydroxyl groups (methoxy groups) increased, the contact angle with respect to a water droplet and the contact angle of water obtained from the contact angle with respect to a bubble on the coating surface tended to decrease. On the other hand, the contact angle hysteresis value of the coating surface tended to increase as the number of methylated hydroxyl groups (methoxy groups) increased.

The polyrotaxane surface exhibits unique behavior in the hydrated state from the viewpoint of the molecular mobility due to the interlocking structure between αCD and the polyethylene glycol chain. For example, the hydration molecular mobility of the polyrotaxane surface was examined from the viewpoint of (1) dissipation energy loss (QCM-D) measured in water using a quartz crystal microbalance with dissipation and (2) the contact angle hysteresis measured by a difference between a contact angle of a water droplet in air and a contact angle of water obtained from a contact angle of bubbles in water. In each coating surface using Examples 1 to 3, the dissipation energy loss and the contact angle hysteresis show a good correlation, and the degree of methylation of the hydroxyl group of the cyclodextrin is considered to be useful for adjusting the molecular mobility. The coating surfaces of Examples 1 to 3 showed a larger contact angle hysteresis value than the DMSO coating surface of Reference Example 1.

3. Differentiation of Myoblasts

(1) Initial Adhesion and Proliferation

Mouse C2C12 myoblasts were seeded on the surface of each culture substrate at a density of 5.0×10³ cells/cm², and proliferated using a medium for proliferation (DMEM, 20% FBS, 100 mg/mL streptomycin, and 1% penicillin) at 37° C. for 4 days in a humidified atmosphere containing 5% CO₂. Cell morphology was recorded using a phase contrast microscope (trade name: IX71, manufactured by Olympus Corporation) including a double CCD digital camera (trade name: DP80, manufactured by Olympus Corporation).

The cultured cells were treated using a trypsin/EDTA solution, and the number of adherent C2C12 cells on each surface was measured at intervals of one day using a hemocytometer.

(2) Evaluation of Myoblast Differentiation

Cells were seeded on the surface of each culture substrate at a density of 7.5×10³ cells/cm², and proliferated using a medium for proliferation at 37° C. for 1 day in a humidified atmosphere containing 5% CO₂. The medium for proliferation was replaced with a medium for differentiation (DMEM, 1% FBS, 100 mg/mL streptomycin, and 1% penicillin).

Adherent cells were observed using a microscope (trade name: IX71, manufactured by Olympus Corporation). On Day 4, whole-cell RNA was extracted from the cells using FastGene RNA Premium Kit.

RNA was reverse transcribed using ReverTra Ace qPCR RT Master Mix (manufactured by TOYOBO CO., LTD.) in a T100 temperature cycler (trade name: BIO-RAD, manufactured by Hercules). The reaction conditions were 37° C. for 15 minutes, 50° C. for 5 minutes, 98° C. for 5 minutes, and then 4° C. for 5 minutes. The gene expression levels of Myog and Myhc with respect to a housekeeping gene (0-actin) were analyzed using THUNDERBIRD SYBR qPCR Mix (manufactured by TOYOBO CO., LTD.) in CFX Connect Real-Time System (trade name: BIO-RAD).

Primers (manufactured by Life Technologies) used in the analysis are as follows.

Primers for Myog:
(SEQ ID NO: 1)
5′-TGGAGCTGTATGAGACATCCC-3′
(SEQ ID NO: 2)
5′-TGGACAATGCTCAGGGGTCCC-3′
Primers for Myhc:
(SEQ ID NO: 3)
5′-CAAGTCATCGGTGTTTGTGG-3
(SEQ ID NO: 4)
5′-TGTCGTACTTGGGCGGGTTC-3′
Primers for ß-actin:
(SEQ ID NO: 5)
5′-GCCTCACTGTCCACCTTCCA-3′
(SEQ ID NO: 6)
5′-AGCCATGCCAATGTTGTCTCTT-3′

PCR cycling conditions were a predenaturation step at 95° C. for 1 minute, followed by 40 cycles of 95° C. for 15 seconds and 60° C. for 1 minute. The gene expression levels of Myog and Myhc with respect to β-actin were calculated. Statistical analysis was performed using one-way analysis of variance (ANOVA) and the Tukey HSD method. Statistical significance was indicated with ***p<0.001, **p<0.01, and *p<0.05. The surface of TCPS (uncoated) was used as a control

### Reference Example 2

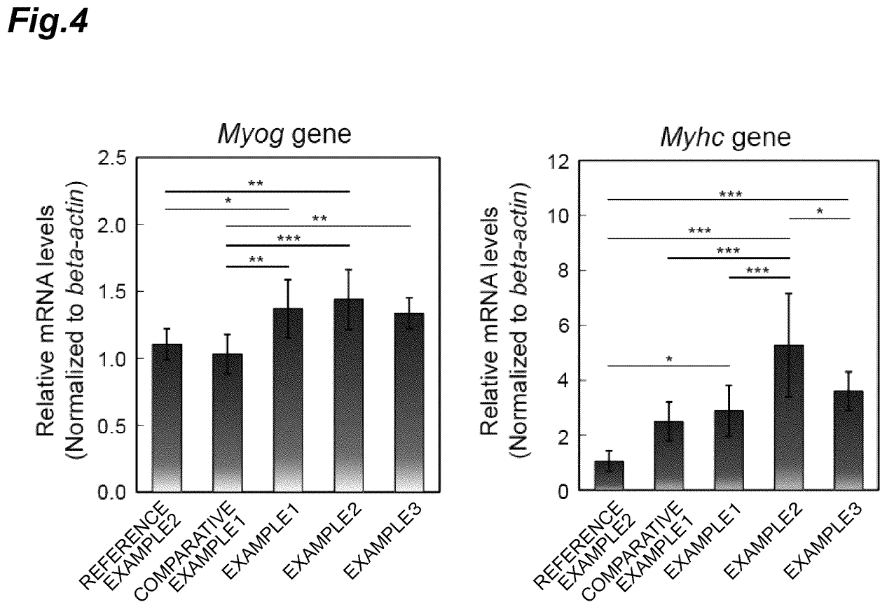

The results are shown in FIG. 4. In the case of culturing using the culture substrate of Comparative Example 1, the expression level of Myog gene was comparable to that in the case of culturing using the culture substrate of Reference Example 2, but the expression level of Myhc gene significantly increased. In the case of culturing using the culture substrates of Examples 1 to 3, the expression levels of both Myog gene and Myhc gene significantly increased. In particular, the increase was significantly large in the culture substrate of Example 2.

3. Morphology and Proliferation of Cells

In order to evaluate the morphology of cells adhered on the substrate surface, BALB/3T3 cells, MC3T3-E1 cells, and MC3T3-G2/PA6 cells were seeded on the surface of the polyrotaxane (Example 3) or the surface of the TCPS (Reference Example 2) at a density of 2.0×10³ cells/cm², and cultured at 37° C. for 24 hours in humidified air containing 5% CO₂ using a medium for proliferation (in the case of BALB/3T3 cells: DMEM, 10% FBS, 100 U/mL penicillin, 100 mg/mL streptomycin; in the case of MC3T3-E1 or MC3T3-G2/PA6 cells: α-MEM, 10% FBS, 100 U/mL penicillin, 100 mg/mL streptomycin). BALB/3T3 cells correspond to fibroblasts, MC3T3-E1 cells correspond to preosteoblasts, and MC3T3-G2/PA6 cells correspond to adipose precursor cells.

Adherent cells were fixed in 4% paraformaldehyde for 10 minutes at room temperature, washed with PBS, and permeabilized with 50 μg/mL digitonin for 5 minutes. For actin filament staining in adherent cells, cells were incubated with Alexa Fluor 555 phalloidin (1:100) (manufactured by Invitrogen) in PBS for 30 minutes at room temperature and washed with PBS. Nuclear DNA was stained with Hoechst 33342 (manufactured by DOJINDO Laboratories) (1:500).

After the cells were washed with PBS, fluorescence images of the stained cells were taken with a fluorescence microscope (trade name: IX71, manufactured by Olympus Corporation) using CellSens standard software (manufactured by Olympus Corporation).

The area and aspect ratio of the diffused cells were analyzed using software ImageJ (manufactured by NIH). The aspect ratio was determined by approximating the shape of the cell to an elliptical shape and then dividing the major axis by the minor axis. At least 50 cells were analyzed for each substrate surface.

In order to evaluate the proliferation of adherent cells on the surface, each cell was cultured in a medium for proliferation for 5 days. The medium was replaced every 3 days. The cell density of the BALB/3T3, MC3T3-E1, or MC3T3-G2/PA6 cells on each surface was determined by counting cells from images taken at intervals of one day over 5 days of culture.

The doubling time of each cell was calculated from the change in the number of adherent cells from 48 hours to 96 hours. Adherent cells were observed using a phase contrast microscope (trade name: IX71, manufactured by Olympus Corporation) equipped with a double CCD digital camera (trade name: DP80, manufactured by Olympus Corporation).

The adhesion areas of the BALB/3T3 cells, the MC3T3-E1 cells, and the MC3T3-G2/PA6 cells on the surface of the polyrotaxane (Example 3) were 2190±1310 μm², 2570±1440 μm², and 6370±4850 μm², respectively. On the other hand, the adhesion areas of the BALB/3T3 cells, the MC3T3-E1 cells, and the MC3T3-G2/PA6 cells on the surface of the TCPS (Reference Example 2) were 1850±900 μm² 2680±1640 μm², and 4170±2300 μm², respectively.

The MC3T3-G2/PA6 cells on the surface of the polyrotaxane (Example 3) extended significantly wider than on the surface of the TCPS (Reference Example 2). At the same time, the aspect ratios of the BALB/3T3 cells, the MC3T3-E1 cells, and the MC3T3-G2/PA6 cells on the surface of the polyrotaxane (Example 3) were 2.7±2.5, 2.9±1.8, and 2.6±1.4, respectively. The aspect ratios of the BALB/3T3 cells, the MC3T3-E1 cells, and the MC3T3-G2/PA6 cells on the surface of the TCPS (Reference Example 2) were 2.4±1.4, 2.3±1.1, and 2.8±1.9, respectively. As described above, the aspect ratio of the MC3T3-E1 cells on the surface of the polyrotaxane (Example 3) was significantly greater than the aspect ratio of the cells on the surface of the TCPS (Reference Example 2).

Next, in order to evaluate the proliferation of the BALB/3T3 cells, the MC3T3-E1 cells, and the MC3T3-G2/PA6 cells, the number of adherent cells on the surfaces of the polyrotaxane (Example 3) and the TCPS (Reference Example 2) was counted every 24 hours.

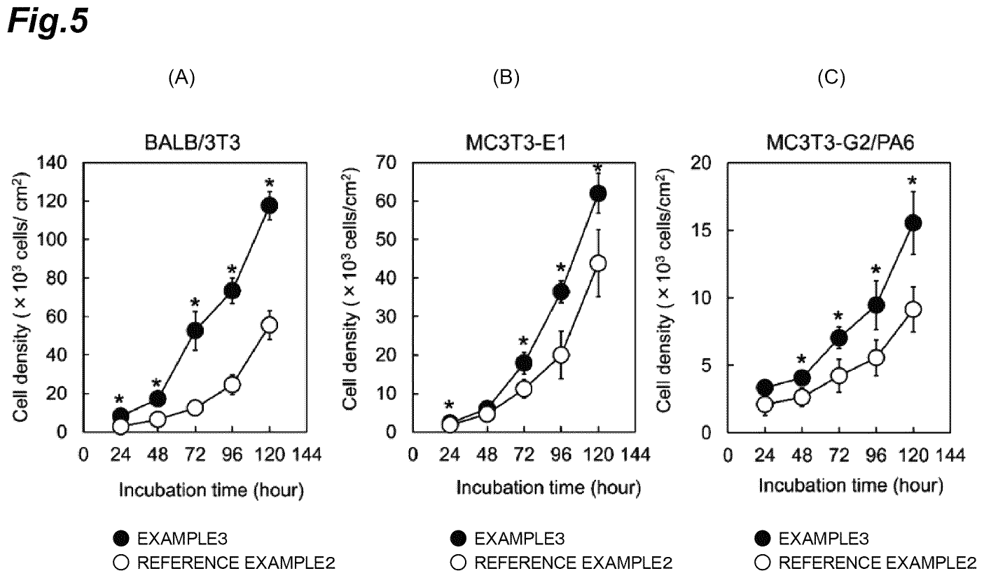

The results are shown in FIG. 5. FIGS. 5(A), 5(B), and 5(C) are graphs showing changes in cell density of BALB/3T3 cells, MC3T3-E1 cells, and MC3T3-G2/PA6 cells, respectively. The number of adherent cells on the surface of the polyrotaxane (Example 3) was significantly greater than the number of cells on the surface of the TCPS (Reference Example 2) regardless of the type of cells. The doubling times of the BALB/3T3 cells, the MC3T3-E1 cells, and the MC3T3-G2/PA6 cells on the surface of the polyrotaxane (Example 3) were 23.0±1.6, 18.5±0.7, and 40.7±6.2, respectively. On the other hand, the doubling times of the BALB/3T3 cells, the MC3T3-E1 cells, and the MC3T3-G2/PA6 cells on the TCPS surface were 25.3±4.0, 24.5±8.2, and 44.6±4.3, respectively. The doubling time of the cells tended to be shorter on the surface of the polyrotaxane (Example 3) than on the surface of the TCPS (Reference Example 2).

4. Differentiation of Cells

In order to induce bone differentiation, MC3T3-E1 cells were seeded on the surfaces of Example 3 and Reference Example 2 at a density of 5.0×10⁴ cells/cm², and cultured in a humidified atmosphere containing 5% CO₂ using a medium for proliferation at 37° C. After 24 hours of incubation, the medium for proliferation was replaced with a medium for bone differentiation (α-MEM, 10% FBS, 100 U/mL penicillin, 100 mg/mL streptomycin, 50 μg/mL magnesium L-ascorbic acid phosphate, and 10 mM disodium β-glycerophosphate pentahydrate). The medium was replaced every 3 days. After MC3T3-E1 cells were cultured in a medium for bone differentiation for 28 days, the adherent cells were washed with PBS and fixed using 99% ethanol at room temperature for 10 minutes.

Alizarin Red S staining was performed to evaluate the calcification action. The fixed cells were washed twice with Milli-Q water and stained with a 1% Alizarin Red S solution for 10 minutes at room temperature. The stained cells were then washed five times with Milli-Q water. Stained cells were photographed using a phase contrast microscope (trade name: IX71, manufactured by Olympus Corporation) equipped with a double CCD digital camera (trade name: DP80, manufactured by Olympus Corporation), and the stained region was quantified using ImageJ software (manufactured by NIH).

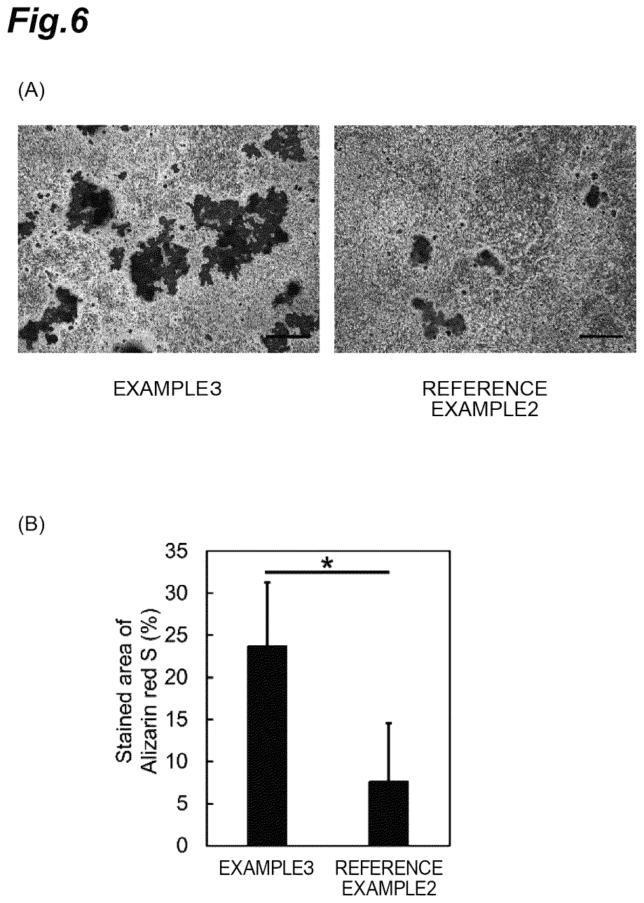

The results are shown in FIG. 6. FIG. 6(A) is an image of cells stained with Alizarin Red S, and FIG. 6(B) is a graph obtained by quantifying a stained region. In the case of culturing on the coating of Example 3, the stained region was significantly increased as compared with the case of culturing on the TCPS of Reference Example 2.

In order to induce adipose differentiation, MC3T3-G2/PA6 cells were seeded on the surfaces of Example 3 and Reference Example 2 at a density of 1.0×10⁴ cells/cm², and cultured in a humidified atmosphere containing 5% CO₂ using a medium for proliferation at 37° C. After 48 hours of incubation, the medium for proliferation was replaced with a medium for adipose differentiation (α-MEM, 10% FBS, 100 U/mL penicillin, 100 mg/mL streptomycin, 0.5 mM IBMX, and 0.25 mM dexamethasone). The medium was replaced every 3 days. After MC3T3-G2/PA6 cells were cultured in a medium for adipose differentiation for 14 days, the cells were washed with PBS and fixed using 4% paraformaldehyde at room temperature for 10 minutes.

An Oil Red O solution (60%, 2-propanol solution) was added to each well to evaluate the accumulation of lipid droplets in the cells. Plates were incubated for 20 minutes at room temperature. The stained cells were then washed twice with PBS. Stained cells were photographed using a phase contrast microscope (trade name: IX71, manufactured by Olympus Corporation) including a double CCD digital camera (trade name: DP80, manufactured by Olympus Corporation), and the stained region was quantified using ImageJ software (manufactured by NIH).

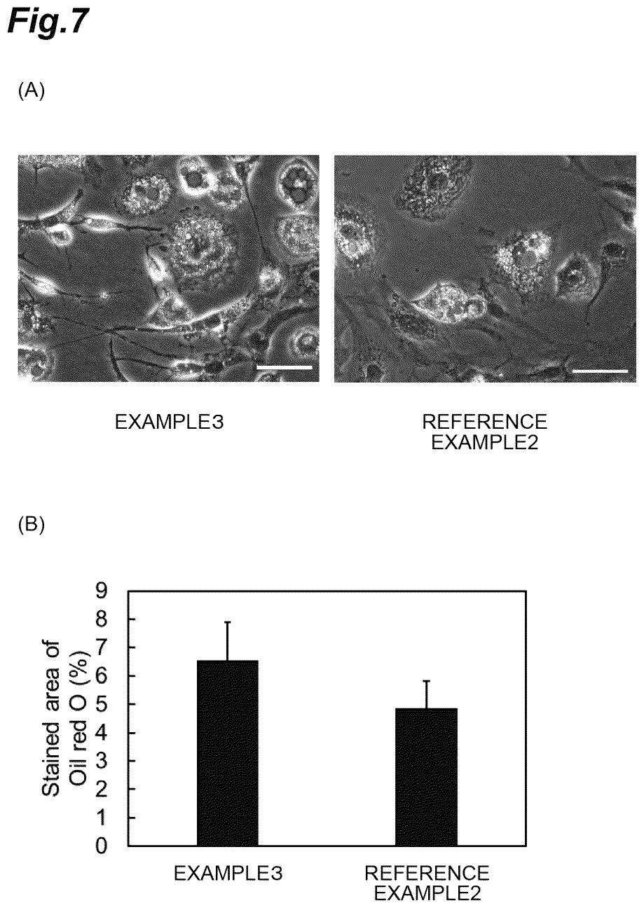

The results are shown in FIG. 7. FIG. 7(A) is an image of cells stained with Oil Red O, and FIG. 7(B) is a graph obtained by quantifying a stained region. In the case of culturing on the coating of Example 3, the stained region tended to increase as compared with the case of culturing on the TCPS of Reference Example 2.

1. A coating composition comprising a polyrotaxane represented by Formula (1): 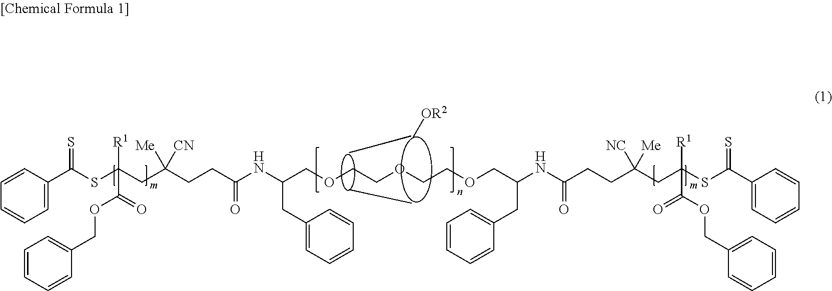
wherein R¹ is a hydrogen atom or a methyl group, m is 1 to 2000, and n is 10 to 500, 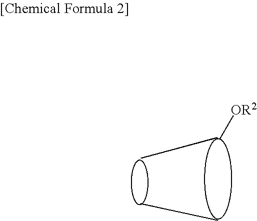
is a cyclodextrin in which at least one hydroxyl group is modified with R², and R² is a C₁₋₆ alkyl group.

2. The coating composition according to claim 1, wherein the polyrotaxane comprises a cyclodextrin modified with 1 to 18 R²s.

3. The coating composition according to claim 1, wherein the polyrotaxane has a number of threaded cyclodextrins of 3 to 220.

4. A cell culture method for promoting differentiation of cells associated with muscle differentiation, bone differentiation, or adipose differentiation, the method comprising:
culturing the cells on a surface of a culture substrate,
the culture substrate including a substrate coated with a composition comprising a polyrotaxane represented by Formula (1): 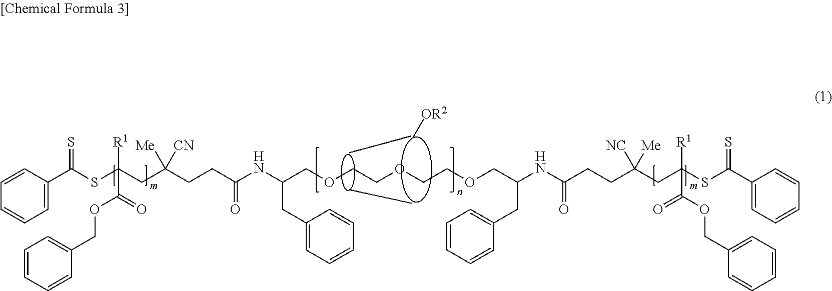
wherein R¹ is a hydrogen atom or a methyl group, m is 1 to 2000, and n is 10 to 500, 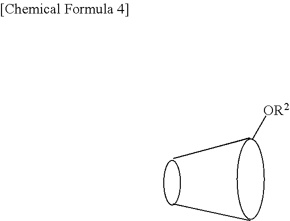
is a cyclodextrin in which at least one hydroxyl group is modified with R², and R² is a C₁₋₆ alkyl group.

5. The method according to claim 4, comprising coating the composition comprising a polyrotaxane represented by Formula (1) onto the surface of the substrate.

6. The method according to claim 4, wherein the polyrotaxane comprises a cyclodextrin modified with 1 to 18 R²s.

7. The method according to claim 4, wherein the polyrotaxane has a number of threaded cyclodextrins of 3 to 220.

8. The method according to claim 4, wherein the cells are preosteoblasts, myoblasts, or adipose precursor cells.

9. The method according to claim 4, wherein differentiation of the cells includes differentiation of myoblasts into myotubes.

10. A culture substrate comprising a substrate coated with a composition comprising a polyrotaxane represented by Formula (1): 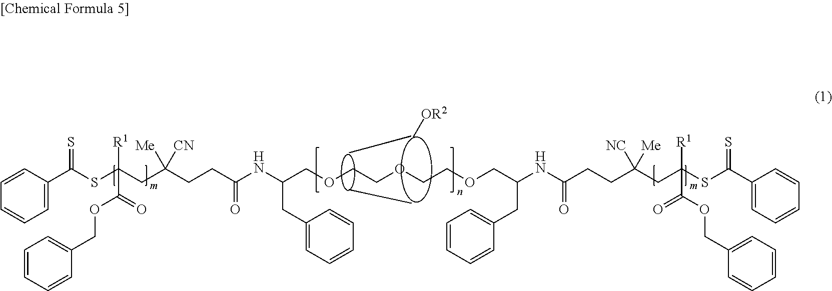
wherein R¹ is a hydrogen atom or a methyl group, m is 1 to 2000, and n is 10 to 500, 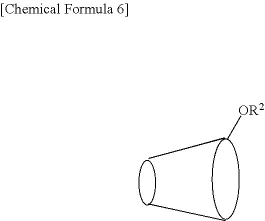
is a cyclodextrin in which at least one hydroxyl group is modified with R², and R² is a C₁₋₆ alkyl group.

11. The culture substrate according to claim 10, wherein the polyrotaxane comprises a cyclodextrin modified with 1 to 18 R²s.

12. The culture substrate according to claim 10, wherein the polyrotaxane has a number of threaded cyclodextrins of 3 to 200.

13. The culture substrate according to claim 10, for promoting differentiation of cells associated with muscle differentiation, bone differentiation, or adipose differentiation.

14. The culture substrate according to claim 13, wherein the cells are preosteoblasts, myoblasts, or adipose precursor cells.

15. The culture substrate according to claim 13, wherein differentiation of the cells includes differentiation of myoblasts into myotubes.

16. The coating composition according to claim 2, wherein the polyrotaxane has a number of threaded cyclodextrins of 3 to 220.

17. The method according to claim 5, wherein the polyrotaxane comprises a cyclodextrin modified with 1 to 18 R²s.

18. The method according to claim 5, wherein the polyrotaxane has a number of threaded cyclodextrins of 3 to 220.

19. The culture substrate according to claim 11, wherein the polyrotaxane has a number of threaded cyclodextrins of 3 to 200.

20. The culture substrate according to claim 11, for promoting differentiation of cells associated with muscle differentiation, bone differentiation, or adipose differentiation.
